(19) United States
(12) Patent Application Publication
(10) Pub. No.: US 2003/0070203 A1
(43) Pub. Date: Apr. 17, 2003

(54) Name card hat

(76) Inventor: Jack Lien, Kaohsiung City (TW)

Correspondence Address: BIRCH STEWART KOLASCH & BIRCH, PO BOX 747, FALLS CHURCH, VA, 22040-0747, US

(21) Appl. No.: 09/976,105

(22) Filed: Oct. 15, 2001

(51) Int. Cl. A61F 9/00

(52) U.S. Cl. 2/12; 2/195.1

(57) ABSTRACT

A name card hat includes a visor blade fastened to a fastening band. The visor blade has folding creases and adjusting apertures formed thereon. The fastening band may engage with the adjusting apertures to pull the visor blade to form an extended and curved sunshade for blocking sunshine. The fastened apertures may be changed to quickly adjust the sunshade angle to block sunshine of different incident angles. The visor blade may be folded and stacked to the size of a name card to facilitate carrying and storing.

### FIELD OF THE INVENTION

The present invention relates to a name card hat and particularly a hat that has a folding visor to facilitate carrying and storing and has an adjustable visor angle to provide sunshade in various sunshine conditions.

### BACKGROUND OF THE INVENTION

Conventional hats have a wide variety of designs and are made of different materials to meet different requirements in various occasions and circumstances. They generally have one common problem and drawback, i.e. the visors are quite bulky in size and are difficult to carry or store. Moreover, the visors are generally fixedly attached to the hat and have a constant shade angle. As a result, the effect of blocking sunshine is impaired when sunshine angle changes at different time.

Furthermore, most hats have a crown to cover people's head that often makes hairs untidy. When worn on the head, the crown forms a closed space and result is poor air ventilation, and often makes wearer's head sweltering in hot seasons. As a result, wearer's hairs often get damped and hairdos are easily spoiled.

### SUMMARY OF THE INVENTION

In view of the foregoing disadvantages, the primary object of the invention is to provide a hat that is thin and compact, and offers a shade to block sunshine and is easy to carry and store.

The hat according to the invention includes a visor blade with folding creases to block sunshine. When extended, the visor blade has tensile ends and a contact rim to form a sunshade. The contact rim has an arched edge to contact wearer's forehead at an anchor point. The tensile ends may be pulled by a fastening band to generate a tension force in a rearward direction to prop up the visor blade. The rearward tension exerting by the tensile ends can prop up the visor blade in the forward direction, and also transfers the force on the contact rim to make the folding creases closing and juxtaposing inwards. As a result, the visor blade is bent in an arched shape and presses against the forehead to form a sunshade for blocking sunshine. When not in use, the visor blade may be folded about the folding creases and stacked to the size of a name card. It is light weight and compact size, and may be carried and stored conveniently.

In addition, the visor blade has adjusting apertures formed thereon below the tensile ends. The fastening band may be engaged with different adjusting apertures to allow the visor blade forming different inclined angles relative to the forehead to block sunshine of different incident angles.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a perspective view of the invention, extended.

FIG. 2 is a pictorial view of the invention, under folding.

FIG. 3 is a pictorial view of the invention, folding to another state.

FIG. 4 is a sectional view of the invention, extended.

FIG. 5 is a fragmentary enlarged side view of the invention.

FIG. 6 is a pictorial view of the invention in use.

FIG. 7 is a schematic view of force analysis of the invention in use.

FIG. 8 is a fragmentary schematic view of the invention, with a closed folding crease.

FIG. 9 is a front view of the invention, with the visor blade extended in an arched form.

FIG. 10 is a side view of the invention.

FIG. 11 is a schematic side view of the invention, at different adjusting conditions.

FIG. 12 is a schematic top view of the invention, with different adjusting curved lines.

FIG. 13 is a schematic view of the invention in use.

FIG. 14 is another schematic view of the invention in use.

FIG. 15 is a schematic view of the invention in use, as an eyeshade.

### DESCRIPTION OF THE PREFERRED EMBODIMENT

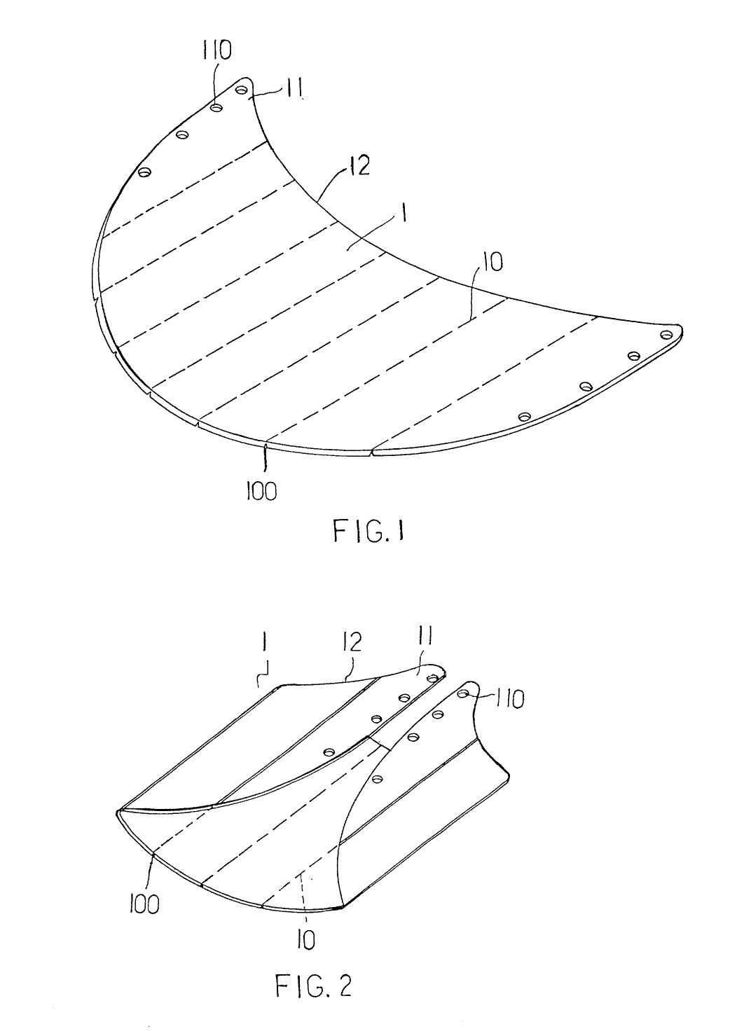

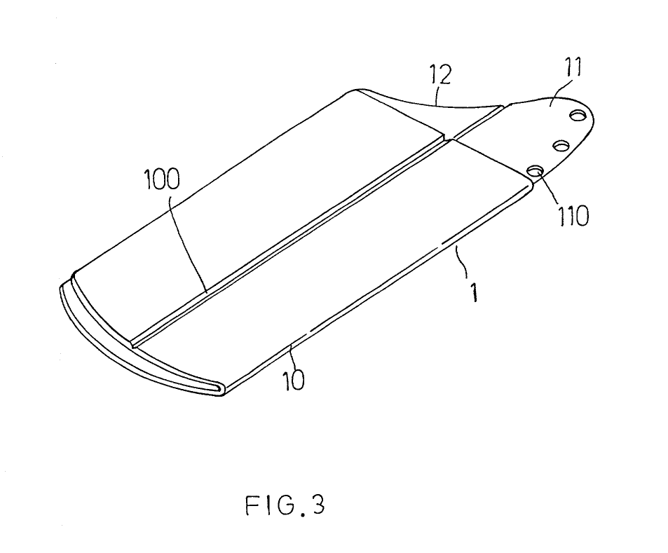

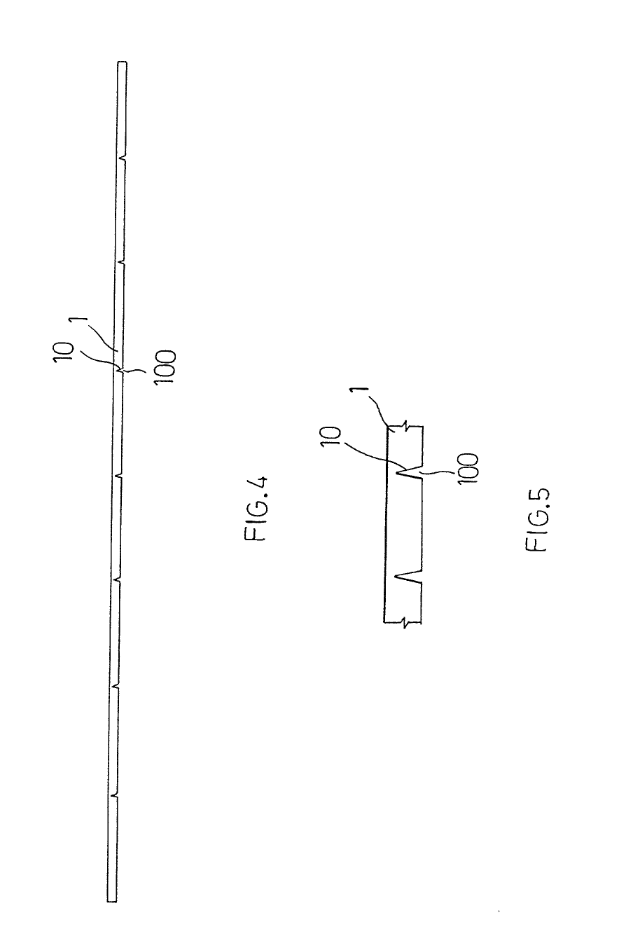

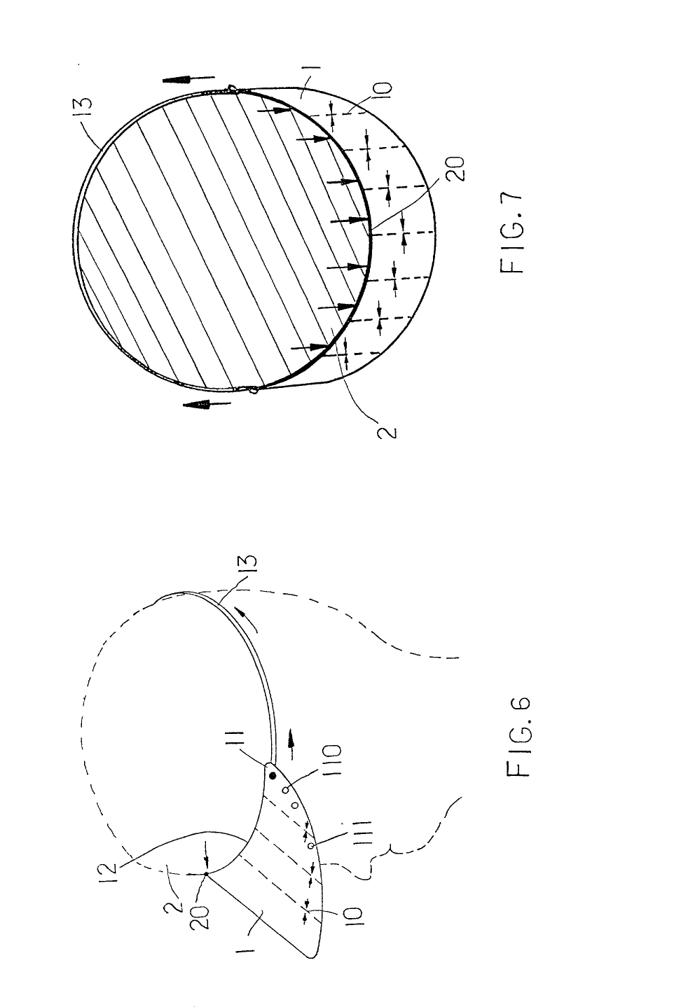

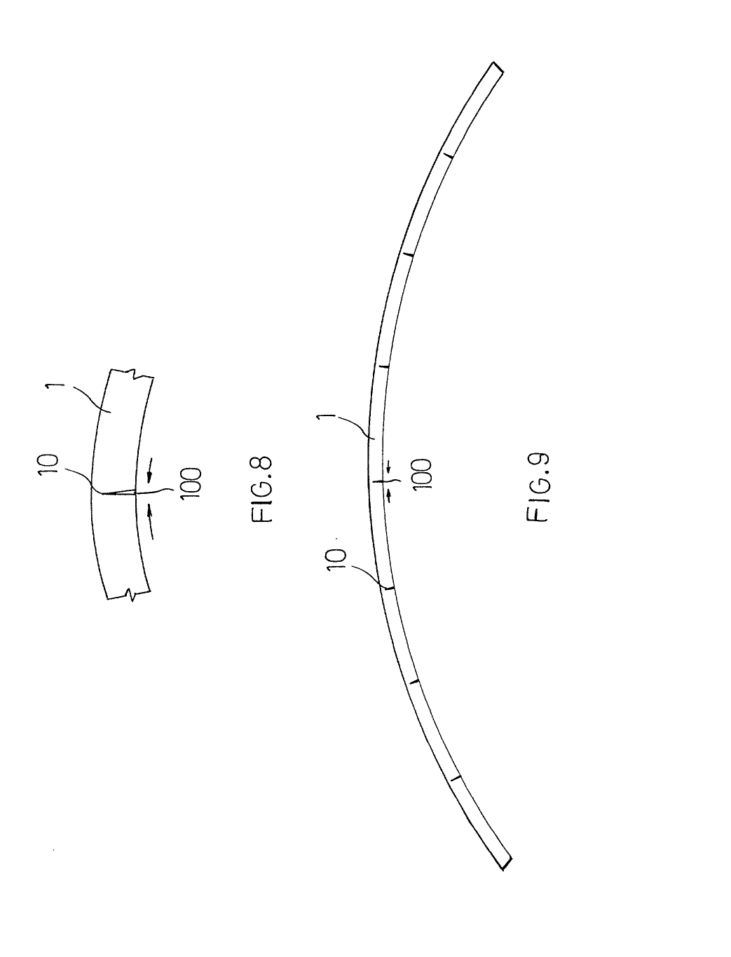

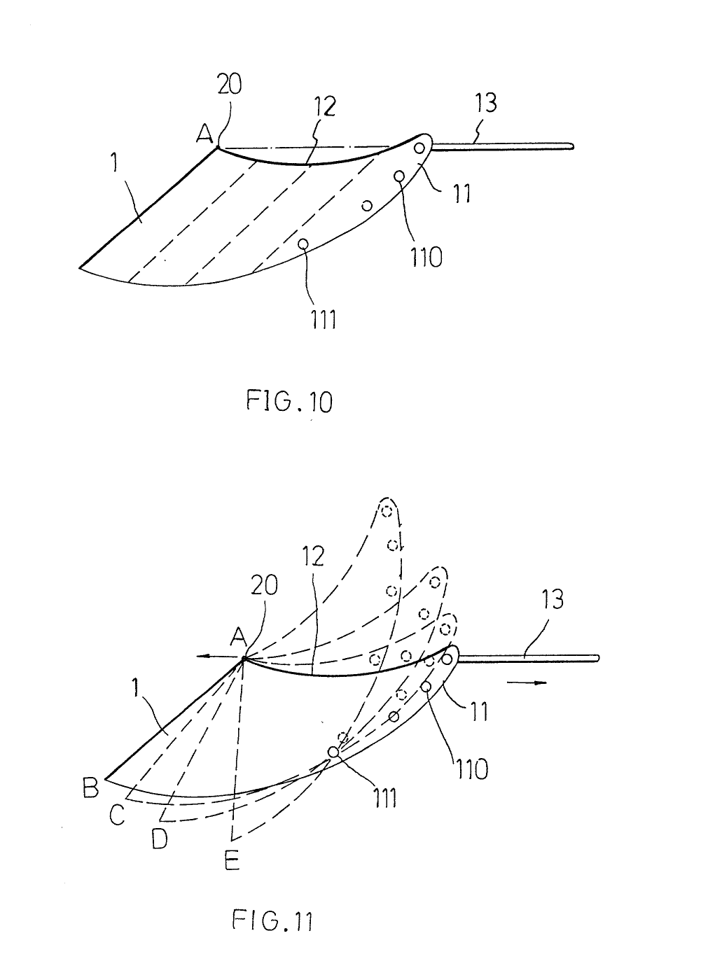

Referring to FIGS. 1, 2 and 3, the invention includes a visor blade 1 which has folding creases 10 formed thereon. The visor blade 1 may be extended as a sunshade to block sunshine (as shown in FIG. 1) or folded and stacked to the size of a name card (as shown in FIGS. 2 and 3). The visor blade 1 has tensile ends 11 located at two sides and a contact rim 12 at one edge thereof. When extended (shown in FIG. 1), the visor blade 1 may be formed a sunshade to block sunshine. There are a plurality of adjusting apertures 110 located on the visor blade 1 at both sides beneath the tensile ends 11. Each of the folding creases 10 has a folding space 100 facing downwards (as shown in FIGS. 4 and 5). The contact rim 12 of the visor blade 1 is curved and may contact wearer's forehead 2 at an anchor point 20. When the tensile ends 11 are fastened to a fastening band 13 and are pulled rearwards, the visor blade 1 is propped up in the forward direction. The rearward pulling force on the tensile ends 11 is transferred through the contact rim (as shown in FIGS. 6 and 7) to make the folding space 100 of the folding creases 10 closed and juxtaposed. As a result, the visor blade 1 will be formed an arched shape through the rearward pulling force of the fastening band 13 to serve as a sunshade for blocking sunshine (shown in FIGS. 8 and 9).

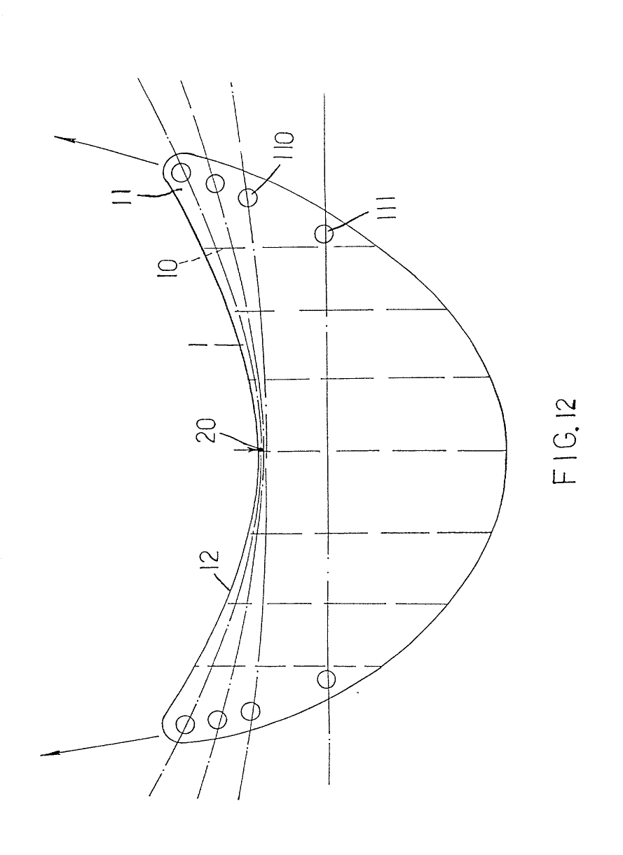

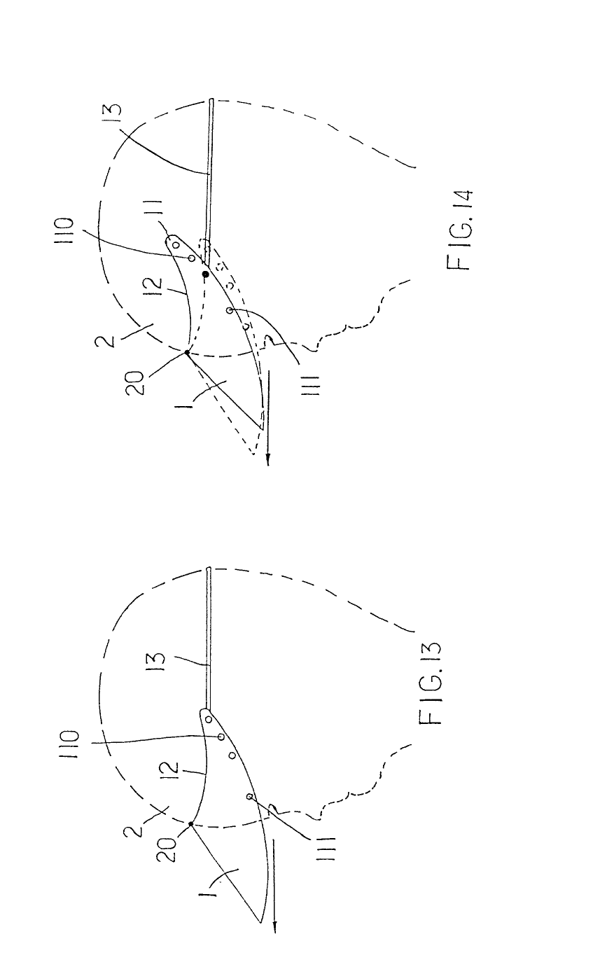

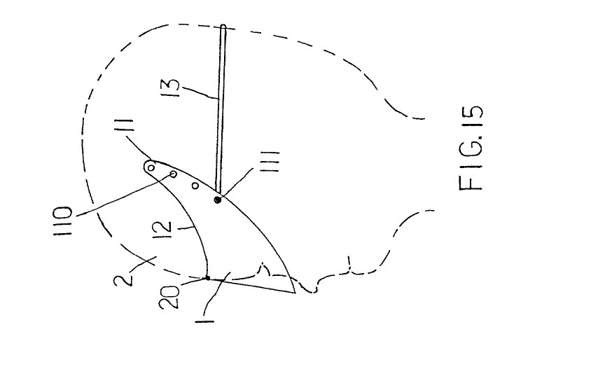

To make the invention to block sunshine of different incident angles (as shown in FIGS. 10, 11 and 12), engage the fastening band 13 with different adjusting apertures 110 to change the curved line angles formed between the apertures 110 and the contact point 20 (shown in FIG. 12). When the visor blade 1 is pulled rearwards by the fastening band 13 against the forehead 2, the visor blade 1 will be tilted to different angles relative to the forehead and point to different spots B, C, D (shown in FIG. 11) thereby to prevent sunshine of different incident angles from reaching wearer's eyes (as shown in FIGS. 13 and 14). When the fastening band 13 is engaged with an adjusting aperture 111 below the contact point 20, the visor blade 1 will be tilted to become an eyeshade (as shown in FIG. 15).

By means of the construction set forth above, and through employing a simple kinetic principle, the visor blade of invention may be formed in a thin and small size, and may be extended to form a curved sunshade to block sunshine of different incident angles to protect wearer's eyes. When not in use, the visor blade may be folded and stacked to the size of a name card to facilitate carrying and storing. In addition, there are other advantages as follows:

1. The invention mainly is focused to protect users' eyes from incident sunshine, and does not affect hairdo. It is light weight and compact, and convenient to carry and use.

2. The invention has adjusting apertures to change sunshade angle to respond and block sunshine of different incident angles. Thus it has greater applicability.

3. Through change of adjusting apertures of fastening, the visor blade may be transformed to an eyeshade to further increase its practical value and usefulness.

What is claimed is:

1. A name card hat, comprising:
a visor blade having two tensile ends located at two sides thereof, a contact rim at an inner edge thereof to contact an user's forehead at an anchor point, a plurality of adjusting apertures formed on two sides of the visor blade beneath the tensile ends, and a plurality of spaced folding creases formed thereon each having a folding space to allow the visor blade folding and stacking to a size of a name card; and
a fastening band having two ends fastened to the adjusting apertures to pull the visor blade rearwards against the forehead to transfer pulling force through the contact rim to close and juxtapose the folding space of the folding crease such that the visor blade is propped up forwards and extended tin a curved shape;
wherein the fastening band is fastened to selected adjusting apertures to change an angle of a curved line formed between the fastened adjusting apertures and the anchor point such that the visor blade is altered to a selected angle to block sunshine of different incident angles.

2. The name card hat of claim 1, wherein contact rim is selectively formed by a curved edge of the visor blade or a plane having a thickness greater than the visor plate flatly bonded to the visor plate.

3. The name card hat of claim 1, wherein the visor blade and the fastening band are selectively made of paper or a soft material.

4. The name card hat of claim 1, wherein the fastening band is selectively fastened to the visor blade by means of rivets, clippers or fasteners.

5. The name card hat of claim 1, wherein the fastening band is selectively extended upwards to form a half crown or a full crown of the hat.

6. The name card hat of claim 1, wherein the folding creases are selectively formed on two sides of the visor blade corresponding to each other or formed on one side of the visor blade with a recess depth same as the thickness of the visor blade.
